(19) United States
(12) Patent Application Publication
(10) Pub. No.: US 2021/0298730 A1
(43) Pub. Date: Sep. 30, 2021

(54) 3D PRINTED SPLINES ON MEDICAL DEVICES AND METHODS TO MANUFACTURE THE SAME

(71) Applicant: Medtronic, Inc., Minneapolis, MN (US)

(72) Inventors: Jonathan Baxter, Fridley, MN (US); Gregory N. Nesseth, Forest Lake, MN (US); Kenneth Warnock, Manchester, MA (US); Kenneth C. Gardeski, Inverness, FL (US)

(21) Appl. No.: 17/215,842

(22) Filed: Mar. 29, 2021

Related U.S. Application Data
(60) Provisional application No. 63/001,832, filed on Mar. 30, 2020.

(51) Int. Cl. A61B 17/00

(52) U.S. Cl. CPC A61B 17/00234; A61B 2017/00247; A61B 2560/04; A61B 2017/00871; A61B 2017/00004; A61B 2017/00292

(57) ABSTRACT

An elongate medical apparatus and a method for manufacturing the same. The apparatus including a base element and at least one spline. The base element and the at least one spline may include various materials to define various characteristics of the apparatus. For example, the base element may include a first material and define a lumen therein. The at least one spline may protrude from an outer surface of the base element and may include a second material. In one or more embodiments, the at least one spline may extend along a helical path.

The present application claims the benefit of U.S. Provisional Application No. 63/001,832, filed Mar. 30, 2020, which is incorporated herein by reference in its entirety.

The disclosure generally relates to medical devices and, in particular, additive manufacturing or 3D printing of medical devices, such as catheters and implantable stimulation leads, including splines.

Medical catheters and leads are commonly used to access vascular and other locations within a body and to perform various functions at those locations, for example, delivery catheters may be used to deliver medical devices, such as implantable medical leads. A number of such medical devices are designed to be navigated through tortuous paths in a human body, such as through a patient's vasculature. Medical catheters and leads may be designed to be sufficiently flexible to move through turns, or curves, in the vasculature yet sufficiently stiff, or resilient, to be pushed through the vasculature. In many cases, such as those involving cardiovascular vessels, the route to the treatment or deployment site may be tortuous and may present conflicting design considerations that may require compromises between dimensions, flexibilities, material selection, operational controls, and the like. Further, the medical catheters and leads may include spline features (e.g., spiraling splines) that are used to advance the medical device within the body and/or to anchor it, either temporarily or permanently. These various properties can present challenges in designing and manufacturing such catheters and leads.

Existing manufacturing processes, such as polymer molding techniques, may provide challenges with forming splines extending from catheters and leads. Further, forming splines from varying materials and having varying shapes and orientations relative to the catheters and leads may also prove challenging using polymer molding techniques.

### SUMMARY

The techniques of the present disclosure generally relate to additive manufacturing of medical devices, such as catheters and leads, that allows for forming one or more splines that extend from an outer surface of the catheter/lead. For example, the one or more splines may be formed using additive manufacturing or three-dimensional (3D) printing. Therefore, the one or more splines may be made of various different materials (e.g., exhibiting various degrees of hardness) that may be the same or different material as the catheter and/or lead upon which the one or more splines are positioned. Further, the one or more splines may extend along a helical path on the outer surface of the catheter and/or lead.

An illustrative elongate implantable medical apparatus may include a base element and at least one spline. The base element many extend along a longitudinal direction between a distal end and a proximal end. The base element may include a first material and may include a lumen therein. The at least one spline may protrude from an outer surface of the base element at least proximate to the distal end only partially covering the outer surface. The at least one spline may include a second material. The first material may define a first Shore durometer that is different than a second Shore durometer of the second material.

In one or more embodiments, the at least one spline may extend along a helical path on the outer surface of the base element.

In one or more embodiments, the at least one spline may include a plurality of splines extending along the longitudinal direction.

In one or more embodiments, the base element may include at least one of a braided wire, inner liner.

An illustrative elongate implantable medical apparatus may include a base element and at least one spline. The base element may extend along the longitudinal direction between a distal end and a proximal end. The base element may include a first material and may define a lumen therein. The at least one spline may protrude from an outer surface of the base element at least proximate to the distal end. The at least one spline may include a second material and the second material may include a bioresorbable material.

In one or more embodiments, the second material may include a steroid material.

In one or more embodiments, the second material may include a drug and a polymer.

In one or more embodiments, the second material may include a drug in a porous silicone material.

An illustrative elongate implantable medical apparatus may include a base element and at least one spline. The base element may extend along the longitudinal direction between a distal end and a proximal end. The base element may include a first material and may define a lumen therein. The at least one spline may protrude from an outer surface of the base element at least proximate to the distal end. The at least one spline may include a second material and the second material may include a steroid material.

An illustrative elongate implantable medical apparatus may include a base element and at least one spline. The base element may extend along a longitudinal direction between a distal end and a proximal end. The base element may define a lumen therein. The at leas one spline may protrude from an outer surface of the base element at least proximate to the distal end. The at least one spline may include a first longitudinal section extending along the longitudinal direction and including a first material, and a second longitudinal section extending along the longitudinal direction and including a second material. The first longitudinal section may be proximate or adjacent to the second longitudinal section.

In one or more embodiments, the at least one spline may extend along a helical path on the outer surface of the base element.

In one or more embodiments, the at least one spline may include a plurality of splines extending along the longitudinal direction.

In one or more embodiments, the first material may define a first Shore durometer that is different than a second Shore durometer of the second material.

An illustrative elongate implantable medical apparatus may include a base element and at least one spline. The base element may extend along a longitudinal direction between a distal end and a proximal end. The base element may include a first portion extending along the longitudinal direction and including a first material, and a second portion extending along the longitudinal direction and including a second material. The first portion may extend proximate or adjacent to the second portion. The base element may define a lumen therein. The at least one spline may protrude from an outer surface of at least one of the first portion and the second portion at least proximate to the distal end.

In one or more embodiments, the at least one spline may extend along a helical path on the outer surface of the base element.

In one or more embodiments, the at least one spline may include a plurality of splines extending along the longitudinal direction.

In one or more embodiments, the first material may define a first Shore durometer that is different than a second Shore durometer of the second material.

In one or more embodiments, the at least one spline may include a first spline and, optionally, a second spline. The first spline may include the first material and may protrude from the outer surface of the first portion. The optional second spline may include the second material and may protrude from the outer surface of the second portion.

In one or more embodiments, the first portion and the second portions may extend along a helical path.

In one or more embodiments, the at least one spline may include a third material that is different than the first and second materials.

An illustrative elongate implantable medical apparatus may include a base element and at least one spline. The base element may extend along a longitudinal direction between a distal end and a proximal end. The base element may define a lumen therein. The at least one spline may protrude from an outer surface of the base element at least proximate to the distal end. The at least one spline may define a fluid flow channel extending along the longitudinal direction. The at least one spline may be configured to protect from tissue intrusion into the fluid flow channel when the apparatus is implanted.

In one or more embodiments, the at least one spline may be configured to deflect toward the outer surface of the base element when the apparatus is implanted such that a surface of the at least one spline may define the fluid flow channel between the at least one spline and the outer surface of the base element.

In one or more embodiments, the at least one spline may define an outermost surface in a radial direction and the fluid flow channel may be defined by a recess in the outermost surface.

In one or more embodiments, the apparatus may also include a hydrophilic material disposed in the recess of the at least one spline.

In one or more embodiments, the apparatus may also include a hydrophilic material disposed on the at least one spline.

An illustrative elongate implantable medical apparatus may include a base element and at least one spline. The base element may extend along a longitudinal direction between a distal end and a proximal end. The base element may define a lumen therein. The at least one spline may protrude from an outer surface of the base element at least proximate to the distal end. The at least one spline may define a spline lumen extending through the at least one spline. The at least one spline may include a flexible material such that the at least one spline is inflatable.

In one or more embodiments, the at least one spline may include a first portion including a first material defining a first Shore durometer and a second portion including the flexible material defining a second Shore durometer less than the first Shore durometer. The second portion may be disposed radially distal to the first portion.

In one or more embodiments, the base element may include a first material defining a first Shore durometer and the flexible material defining a second Shore durometer less than the first Shore durometer.

An illustrative elongate implantable medical apparatus may include a base element and at least one spline. The base element may extend along a longitudinal direction between a distal end and a proximal end. The base element may define a lumen therein and the base element may include a flexible material such that the base element is inflatable. The at least one spline may protrude from an outer surface of the base element at least proximate to the distal end. The at least one spline may include a flexible material such that the at least one spline is expandable.

An illustrative elongate implantable medical apparatus may include a base element and at least one spline. The base element may extend along a longitudinal direction between a distal end and a proximal end. The base element may define a lumen therein. The at least one spline may protrude from an outer surface of the base element at least proximate to the distal end. The at least one spline may include a first spline extending along a first helical path and a second spline extending along a second helical path extending in an opposite rotational direction than the first helical path such that the first spline and the second spline intersect.

In one or more embodiments, the first spline may protrude from the outer surface in a radial direction by a first distance greater than a second distance the second spline protrudes from the outer surface.

In one or more embodiments, the second spline may protrude from the outer surface in a direction towards the proximal end.

In one or more embodiments, the first spline may include a first material and the second spline may include a second material. The first material may define a first Shore durometer that is less than a second Shore durometer of the second material.

An illustrative method for additive manufacturing of an implantable medical catheter may include feeding a substrate through a substrate channel in a heating cartridge. The substrate channel in fluid communication with an interior cavity of the heating cartridge. Also, the method may include feeding at least a first filament through a filament port into the interior cavity, melting the first filament in the interior cavity, and moving the heating cartridge relative to the substrate at least in a longitudinal direction and a first rotational direction to form a catheter jacket and a first spline protruding from an outer surface of the catheter jacket comprising material from the first filament. The first spline may extend along a first helical path. Further, the method may include moving a funnel element relative to the substrate at least in a longitudinal direction and a second rotational direction opposite to the first rotational direction to form a second spline protruding from the outer surface of the catheter jacket comprising material from the first spline. The second spline may extend along a second helical path extending in an opposite rotational direction than the first helical path such that the first spline and the second spline intersect.

An illustrative elongate implantable medical apparatus may include a base element and at least one spline. The base element may extend along a longitudinal direction between a distal end and a proximal end. The base element may define a lumen therein. The at least one spline may protrude from an outer surface of the base element at least proximate to the distal end. The at least one spline may define a textured outermost surface to facilitate implantation.

In one or more embodiments, the textured outermost surface may define a plurality of dimples.

In one or more embodiments, the textured outermost surface may define a cross-hatch.

In one or more embodiments, the textured outermost surface may define a plurality of microgrooves.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 illustrates a side view of an elongate medical apparatus including one or more splines extending from an outer surface of the elongate medical apparatus according to the present disclosure.

FIG. 2A is a cross-sectional view of an illustrative embodiment of the elongate medical apparatus of FIG. 1.

FIG. 2B is a cross-sectional view of another illustrative embodiment of the elongate medical apparatus of FIG. 1.

FIG. 3 illustrates a side view of another illustrative embodiment of an elongate medical apparatus having splines formed from two different materials.

FIG. 4 illustrates a side view of another illustrative embodiment of an elongate medical apparatus having two different materials.

FIG. 5 illustrates a side view of another illustrative embodiment of an elongate medical apparatus having two different materials and including splines formed from the corresponding different materials.

FIG. 6 illustrates a side view of another illustrative embodiment of an elongate medical apparatus having two different materials.

FIG. 7A is a cross-sectional view of an elongate medical apparatus defining one or more flow channels between splines.

FIG. 7B is a cross-sectional view of an elongate medical apparatus defining one or more flow channels between a spline and an outer surface of the elongate medical apparatus.

FIG. 7C is a cross-sectional view of an elongate medical apparatus defining one or more flow channels in one or more splines.

FIG. 8A is a cross-sectional view of an elongate medical apparatus including one or more splines defining a lumen therein.

FIG. 8B illustrates another embodiment of an elongate medical apparatus including one or more splines defining a lumen therein.

FIG. 8C is a cross-sectional view of another elongate medical apparatus including one or more splines defining a lumen therein.

FIG. 8D is an expanded cross-sectional view of yet another elongate medical apparatus including one or more splines embedded in a base element and defining a lumen therein.

FIG. 9 illustrates a side view of an elongate medical apparatus including two splines extending along opposing helical paths.

FIG. 10 is a cross-sectional view of another illustrative embodiment of the elongate medical apparatus of FIG. 9.

FIG. 11 illustrates an outer surface of the elongate medical apparatus of FIG. 9 projected along a plane.

FIG. 12A illustrates a side profile of a first spline of the elongate medical apparatus of FIG. 9 flattened onto a horizontal plane.

FIG. 12B illustrates a side profile of a second spline of the elongate medical apparatus of FIG. 9 flattened onto a horizontal plane.

FIG. 13 is a cross-sectional view of an elongate medical apparatus including one or more splines having a textured outermost surface.

FIG. 14 is a flow diagram that illustrates one example of a method for additive manufacturing of an elongate medical apparatus of FIGS. 9-11.

FIG. 15 illustrates an outer surface of an elongate medical apparatus including two crossing splines projected along a plane.

### DETAILED DESCRIPTION

The present disclosure generally provides additive manufacturing systems and methods for medical devices, such as catheters and leads, that provides one or more splines extending from an outer surface of the medical device. The one or more splines may be configured in a variety of different ways depending on the application. For example, the one or more splines may be formed from a different or same material as the remainder of the medical device. Also, for example, each spline of the one or more splines (or portions of each spline) may be made from one or more materials. Specifically, the different or same materials may define various characteristics (e.g., hardness). Further, for example, the one or more splines may include bioresorbable materials or drug materials. In one or more embodiments, the one or more splines may include a hydrophilic material to assist in advancing the medical device within the patient.

The one or more splines may be formed on the medical device using additive manufacturing techniques, which may, for example, be described as three-dimensional (3D) printing. The additive manufacturing techniques may allow for the one or more splines to be formed on the medical device in a way such that the one or more splines include the characteristics and features described herein (e.g., different materials, configurations, etc.).

For example, a base element of the medical device (e.g., a catheter jacket) and the one or more splines, which extend or protrude from the base element, may include (e.g., be formed of) various materials that may be the same or different from each other. Further, a portion of the base element may be the same or different material as another portion of the base element. Also, a portion of the one or more splines may be the same or different materials as another portion of the one or more splines. Therefore, the medical device (e.g., including the one or more splines) may be customizable or optimizable for a specific application depending on characteristics of the materials used to form one or both of the base element and the splines.

Specifically, in one embodiment, the materials forming the base element may define a hardness that is different than a material forming the one or more splines. In other words, different portions of the base element and the splines may define a softer (e.g., rigid resilient) or harder (e.g., stiffer) material. As such, the material of base element may define characteristics for a specific anatomy requirement (e.g., pushability, flexibility, torque transfer, etc.) and the material of the splines may define characteristics for advancing the shaft or anchoring it in a specific body tissue. Therefore, the characteristics of the device may be specifically customized to a particular application.

In one or more embodiments, the medical device may include sections of alternating materials (e.g., having different characteristics) produced through additive manufacturing techniques. For example, the medical device may alternate between relatively softer and relatively harder sections of the medical device. Specifically, one or both of the base element and the one or more splines may alternate materials in subsequent sections. For example, the base element may alternate materials at segmented sections while the one or more splines is formed from a single material. Further, for example, the base element may be a single material while the one or more splines alternate materials at segmented sections. Still further, for example, both the base element and the one or more splines may alternate materials (e.g., such that material of the base element and the one or more splines is consistent at the same location) at segmented sections. In one or more embodiments, the segmented sections may be defined along a helical path. The sections (e.g., oriented in a “zebra-stripe pattern”) may assist in increasing the shaft flexure while allowing a stiffer spline to transfer torque.

As used herein, the term “or” refers to an inclusive definition, for example, to mean “and/or” unless its context of usage clearly dictates otherwise. The term “and/or” refers to one or all of the listed elements or a combination of at least two of the listed elements.

As used herein, the phrases “at least one of” and “one or more of” followed by a list of elements refers to one or more of any of the elements listed or any combination of one or more of the elements listed.

As used herein, the terms “coupled” or “connected” refer to at least two elements being attached to each other either directly or indirectly. An indirect coupling may include one or more other elements between the at least two elements being attached. Either term may be modified by “operatively” and “operably,” which may be used interchangeably, to describe that the coupling or connection is configured to allow the components to interact to carry out described or otherwise known functionality. For example, a controller may be operably coupled to a resistive heating element to allow the controller to provide an electrical current to the heating element.

As used herein, any term related to position or orientation, such as “proximal,” “distal,” “end,” “outer,” “inner,” and the like, refers to a relative position and does not limit the absolute orientation of an embodiment unless its context of usage clearly dictates otherwise.

All scientific and technical terms used herein have meanings commonly used in the art unless otherwise specified. The definitions provided herein are to facilitate understanding of certain terms used frequently herein and are not meant to limit the scope of the present disclosure.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

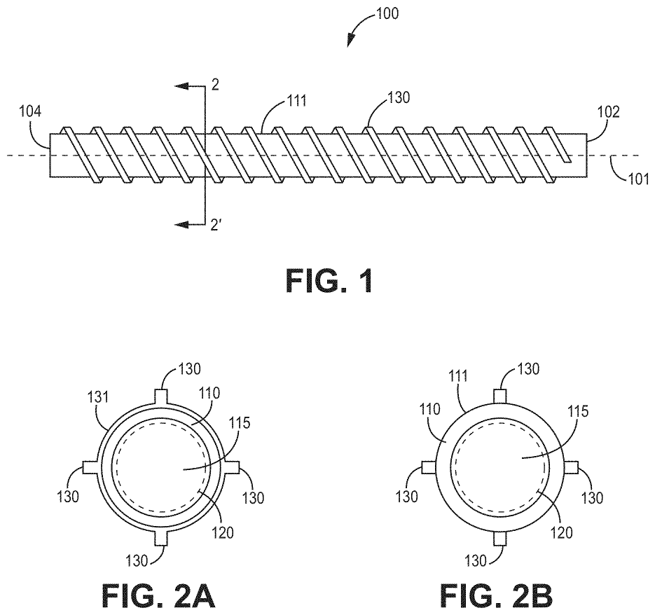

FIG. 1 shows one example of an elongate implantable medical apparatus, or device, 100 including a base element 110 (e.g., a catheter jacket) having at least one spline 130 protruding from the base element 110. The base element 110 may define the basic structure of the apparatus 100 and may extend along a longitudinal direction 101 (e.g., a path or axis along which the base element 110 extends) between a distal end 104 and a proximal end 102. It is noted that the longitudinal direction 101 may extend along a path that is curved in two or three dimensions (e.g., to form a medical apparatus 100 following the same path) but is shown as extending along a straight longitudinal axis in FIG. 1. The base element 110 may define any suitable shape and size, and may include any suitable components in forming the desired medical apparatus 100 (e.g., electrical components, conductive materials, markers, steering elements such as pullwires or pullwire termination bands, structural elements, release mechanisms, etc.). For example, in one or more embodiments, electrical components may include sensors (e.g., pressure sensors), antennae, inductors, infrared diodes, diode lasers, laser waveguides, electrical shunts, etc. Also, for example, markers may include radiopaque materials (e.g., metal and non-metal, platinum, tantalum, tungsten, barium sulfate, bismuth, etc.) or an RLC circuit forming a harmonic oscillator that may be tuned to an MRI scanner as a beacon for image-guided therapies. In one or more embodiments, the structural elements may include a braid or a coil, and the release mechanisms may include wire or tether including surgical suture or UHMWPE fiber (DSM Dyneema®). Additionally, the base element 110 may define a lumen 115, or elongate opening, therein (e.g., extending within the base element 110 along the longitudinal direction 101), e.g., as shown in cross-sectional views illustrated in FIGS. 2A-2B. The lumen 115 may be configured such as those known in the catheter art.

The base element 110 may include (e.g., be formed of) any suitable material. For example, in one or more embodiments, the base element 110 may include a polymer material. Specifically, the base element 110 may include a thermoplastic material, a thermoset polymeric material, thermoplastic polyurethanes, nylons, nylon copolymers (e.g., Arkema Pebax® which may include block copolymers including rigid polyamide blocks and soft polyether blocks), polyethylene teraphthalate, polybutylene teraphthalate, polyvinylidene fluoride, Chronoprene®, butyl rubber (e.g., a copolymer of isobutylene and isoprene), high density polyethylene, silicone, polyimide, conductive polymers (e.g., such as PEDOT, poly(3,4-ethylenedioxythiophene), or thermoplastics with high loadings of carbon black), etc., or combinations thereof. In one or more embodiments, nylons may include, for example, polyamides, PA-12, PA-6, PA-6/6, with or without fillers, and the nylons may be reinforced with glass fibers, glass beads, and carbon fibers to improve their mechanical and thermal performance.

In one or more embodiments, the apparatus 100 may further include a support element 120 positioned, or located, within the lumen 115 of the base element 110 to provide a structure upon which the base element 110 may be positioned or constructed. The support element 120 may also be described as providing shaft reinforcement to, e.g., transfer torque of the apparatus 100 when in use. The support element 120 may extend along the longitudinal direction 101 between the distal end 104 and the proximal end 102. The support element 120 may be optional and is shown in broken lines in FIGS. 2A-2B. In one or more embodiments, the support element 120 may include a liner, a wire (e.g., braided), a polymer filament, or any other type of reinforcement. In one or more embodiments, the liner may include PTFE, HDPE, PVDF, and/or Pebax®. Further, the liner may utilize a lubricious coating that includes silicone oil and/or reactive silicone lubricant. Further, in one or more embodiments, the braid wire may include Type 304 stainless steel with spring temper, which may be optimized for the indicated use. Also, for example, the polymer filament may define a higher melt temperature than the base element 110 and may be braided or coiled to reinforce the shaft. Additionally, in one or more embodiments, the support element 120 may include hybrid metal/fabric braided coils such as described in U.S. Pat. No. 10,080,862, entitled “TUBULAR BODIES FOR MEDICAL DELIVERY DEVICES AND RELATED MANUFACTURING METHODS,” which is herein incorporated by reference. In such embodiments in which the apparatus 100 defines a lumen, the lumen 115 may be defined within the support element 120. In one or more embodiments, the support element 120 may be permanent (e.g., included in the resultant apparatus 100) or may be removable (e.g., after forming the apparatus 100).

The apparatus 100 may also include at least one spline 130 protruding from an outer surface 111 of the base element 110. The at least one spline 130 may be located at least proximate to the distal end 104 of the base element 110. The at least one spline 130 may assist in advancing the medical apparatus, or device, 100 within the body of a patient and/or to anchor the medical apparatus 100, either temporarily or permanently. The at least one spline 130 may extend for any suitable length along the base element 110. For example, the at least one spline 130 may extend along the longitudinal axis 101 for a distance for which the apparatus 100 may exit an access catheter, backup catheter or introducer sheath. In one or more embodiments, the length of the at least one spline 130 extending along the longitudinal axis 101 may be determined as to limit the amount of friction that may be created between the at least one spline 130 and the tissue when implanted (e.g., to prevent inhibiting advancement of the catheter or lead).

As shown in FIG. 1, the at least one spline 130 may extend along the longitudinal direction 101 between the proximal end 102 and the distal end 104. In other embodiments, the at least one spline 130 may extend along a distal portion or length of the base element 110 (e.g., only proximate the distal end 104). The distance by which the at least one spline 130 extends from the distal end 104 may be, for example, greater than or equal to about 0.125 inches (e.g., 0.3175 cm), greater than or equal to about 0.1875 inches (e.g., 0.47625 cm), greater than or equal to about 0.250 inches (e.g., 0.635 cm), etc. and/or less than or equal to about 2.50 inches (e.g., 6.35 cm), less than or equal to about 1.5 inches (e.g., 3.81 cm), less than or equal to about 1.00 inch (e.g., 2.54 cm), etc. Additionally, the distance by which the at least one spline 130 extends from the distal end 104 may be a ratio of the spline length (e.g., along the longitudinal axis) to the base element 110 diameter. In one or more embodiments, the spline 130 may define a shorter length for base element 110 diameter of, e.g., about 3-7 Fr (French scale) and the spline 130 may define a longer length for base element 110 diameter of, e.g., about 7-30 Fr. In one or more embodiments, the distance by which the at least one spline 130 extends from the distal end 104 may be a percentage of the entire length of the medical apparatus 100 of, for example, about 0.5% on a septal vein pacing lead or a CRT pacing lead, about 3% on a long interventional catheter, a septal vein lead delivery catheter or a CRT lead delivery catheter, or about 25% on a lead delivery catheter for an extra-vascular ICD lead. In yet other embodiments, the at least one spline 130 may extend along the base element 110 in discrete segments between the proximal end 102 and the distal end 104.

The length of the at least one spline 130 may depend on the indicated use, resulting in various different desired ratios of spline length to base element diameter. For example, the ratio may be variable, resulting in an envelope on a graph depicting spline longitudinal length and the base element diameter. Specifically, in some applications, the device 100 may traverse various sharp turns, therefore, the at least one spline 130 may include multiple spline segments (e.g., as compared to one longer spline segment starting at the distal end 104) along the longitudinal direction to provide improved performance in reaching a target location in the anatomy. As the splines engage tissue of the anatomy, the splines may also create friction that may be overcome in order to rotate-to-advance. In such embodiments, multiple spline segments may help to “pull” the catheter delivery system along at different longitudinal locations of the shaft. As such, the material section of the splines may help keep the friction down so there is a balance between coefficient of friction, spline height and pitch. Further, lubricious coatings (e.g., hydrophilic coatings) may assist in optimizing the friction.

In one or more embodiments, the at least one spline 130 may only partially cover the outer surface 111 of the base element 110. For example, as shown in the cross-sectional view of FIG. 2B, the at least one spline 130 may protrude from the base element 110 at distinct radial locations such that the base element 110 (e.g., the outer surface 111) is exposed to external environment between each spline of the at least one spline 130.

In other embodiments, the at least one spline may include a spline layer 131 that covers the base element 110. For example, as shown in the cross-sectional view of FIG. 2A, the spline layer 131 may extend between each spline of the at least one spline 130 (e.g., such that the base element 110 may not be exposed to external environment at the location of the spline layer 131). Depending on the manufacturing process of the medical apparatus 100, the spline layer 131 may be present between the splines 130 (e.g., as shown in FIG. 2A) or the spline layer 131 may not be present between the splines 130 (e.g., as shown in FIG. 2B). For example, if the spline material is the same or similar to the base element material, no spline layer 131 may be present (e.g., as shown in FIG. 2B). Also, for example, for polymers with poor miscibility, the apparatus 100 may include a spline layer 131, e.g., with some type of surface preparation or tie layer (e.g., as shown in FIG. 2A).

The medical apparatus 100 may be manufactured in any suitable way. For example, the base element 110 may be constructed using additive manufacturing as described in U.S. Pat. App. No. 62/927,092, entitled “ADDITIVE MANUFACTURING FOR MEDICAL DEVICES,” which is herein incorporated by reference. Further, the at least one spline 130 may similarly be manufactured using an additive process. Specifically, in one or more embodiments, the base element 110 and the at least one spline 130 may be formed during a single pass of a heating cartridge, which heats one or more filament materials and forms the materials into the desired shape (e.g., of the base element 110 and the at least one spline 130). In other embodiments, the base element 110 may be formed during a first pass of the heating cartridge and the at least one spline 130 may be formed during a second pass of the heating cartridge (e.g., without moving or manipulating the base element 110 before the second pass), which may be described as a one setup process. In one or more embodiments, the heating cartridge may include an extrusion die of static or dynamic profile that forms filament materials to the desired catheter jacket profile.

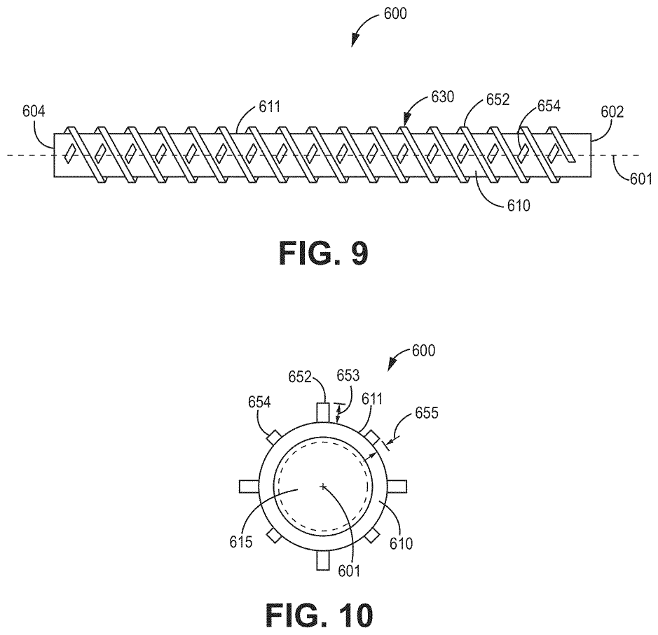

Further, in one or more embodiments, the catheter segments on which the splines 130 are formed may be extruded at a larger outside diameter that could then be laser ablated to result in splines, cross-splines, etc. protruding from the same base outside diameter as the non-spline segment (e.g., the base element 110). Also, for example, in forming cross-splines (e.g., as shown in FIG. 9), a fused deposition modeling type nozzle with a radius profiled slotted spout may be used to quickly form standalone spline segments. In one or more embodiments, an imprint wheel (e.g., including semi-circle cut-outs) may be used to displace still melted filament material to form splines, cross-splines, etc. Additionally, in one or more embodiments, a skiving blade may be used to displace still melted (or semi-melted) filament material to form cross-splines, standalone splines, etc.

The at least one spline 130 may extend along the longitudinal direction 101 along any suitable path. For example, as shown in FIG. 1, the at least one spline 130 extends along a helical path on the base element 110. In other words, the at least one spline 130 may corkscrew or spiral around the base element 110 while traversing the base element 110 along the longitudinal direction 101. The at least one spline 130 may define any suitable pitch along the longitudinal direction 101 (e.g., a distance between each coiling of the spline). Specifically, the pitch of the at least one spline 130 may depend on the application of the apparatus 100. In one or more embodiments, the pitch of the at least one spline 130 may depend on a diameter of the base element 110. For example, the pitch of the at least one spline 130 may be about one to three times the diameter of the base element 110. In one or more embodiments, the at least one spline 130 may extend parallel to the longitudinal direction 101 or asymmetrically along the apparatus 100.

The at least one spline 130 may include any number (e.g., a plurality) of splines (e.g., portions of a contiguous spline). For example, as shown in FIGS. 2A and 2B, the at least one spline 130 includes four splines. The splines 130 may be spaced around the apparatus 100 in any suitable way (e.g., such that the splines 130 are equidistant from one another, such that the splines 130 have varying distance between one another, such that the splines 130 have at least one different distance between one another, etc.).

The at least one spline 130 may include (e.g., be formed of) any suitable material. For example, in one or more embodiments, the at least one spline 130 may include a polymer material. Specifically, the at least one spline 130 may include a thermoplastic material, a thermoset polymeric material, a bioresorbable material, a steroid, a hydrophilic coating, a heparin coating (e.g., to manage the average clotting time), etc., or combinations thereof.

It may be described that the base material 110 includes (e.g., is formed of) a first material and the at least one spline 130 includes (e.g., is formed of) a second material, where the first material is different from the second material. In one or more embodiments, the first material (of the base element 110) may define a first Shore durometer that is different than a second Shore durometer of the second material (of the at least one spline 130). For example, the first material may define a first Shore durometer that is less (e.g., softer) than a second Shore durometer of the second material. As such, the base element 110 may be softer than the at least one spline 130 (e.g., such that the base element 110 defines the appropriate characteristics of pushability, flexibility, and/or torque transfer and the at least one spline 130 defines the appropriate characteristics for advancing and/or anchoring). Also, in one or more embodiments, the first material may define a first Shore durometer that is greater (e.g., harder) than a second Shore durometer of the second material.

In one or more embodiments, the material forming one or both of the base element 110 and the at least one spline 130 may include a bioresorbable material. In one or more embodiments, the material forming one or both of the base element 110 and the at least one spline 130 may include a steroid material. In one or more embodiments, the material forming one or both of the base element 110 and the at least one spline 130 may include a drug and a polymer. In one or more embodiments, the material forming one or both of the base element 110 and the at least one spline 130 may include a drug in a porous silicone material. For example, the at least one spline 130 may include a specific drug to silicone composition such that the drug may be released into the surrounding tissue in a controlled manner.

Each of these additional materials may be disposed on the apparatus 100 in any suitable way. For example, the medical apparatus 100 may be constructed using additive manufacturing as described in U.S. Pat. App. No. 62/927,092, entitled “ADDITIVE MANUFACTURING FOR MEDICAL DEVICES,” which is herein incorporated by reference. Therefore, the additional material (e.g., bioresorbable material, steroid material, drug, etc.) may be printed on the medical apparatus 100 by, e.g., pixel-by-pixel 3D printing, using multiple print heads, through a multi-layer process, etc.

Bioresorbable material may be used on splines of a sleevehead of a cardiac pacing lead with spiral splines to provide additional fixation with active fixation leads, which may facilitate screwing the lead deeper into the myocardium or to anchor a lead deep in the coronary veins such as the septal vein. Sleeveheads may be molded polyurethane components located between the distal cathode and the anode ring. The bioresorbable material may be incorporated the spiral fixation splines, for example, to facilitate removability of the lead. Segments of the spiral splines, which may be described as intermittent spiral splines or “nubs,” may include (e.g., be formed of) the bioresorbable material to increase fixation during implantation. Bioresorbable spiral spline material may include a steroid and, the sleevehead may also include steroid integrated (e.g., 3D printed) into the sleevehead itself, to create an integrated monolithic controlled release device (MCRD).

Specifically, in one or more embodiments, the at least one spline 130 including a steroid material may include porous silicone, which may be impregnated with dexamethasone acetate and may be referred to as a monolithic controlled release device (MCRD). In other embodiments, the at least one spline 130 may include porous polyurethane and other steroids like dexamethasone sodium phosphate and beclomethasone. These configurations may elute steroid along an exponentially decaying elution curve for years. Additionally, the porous carrier material may remain even when the elution is immeasurable (e.g., after the elution has completely eluted from the carrier).

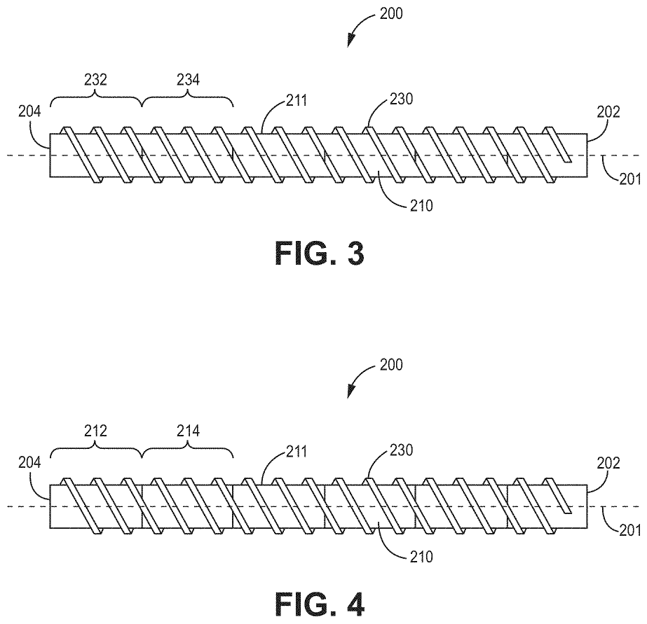

As shown in FIGS. 3-5, the medical apparatus 200 may include segmented sections or portions of alternating or varying materials. The medical apparatus 200 may include the same components and characteristics as described pertaining to medical apparatus 100 illustrated in FIGS. 1-2. For example, the medical apparatus 200 may include a base element 210 extending along a longitudinal direction 201 between a distal end 204 and a proximal end 202, and the base element 210 may define a lumen therein. Also, similar to medical apparatus 100 illustrated in FIGS. 1-2, the medical apparatus 200 may include at least one spline 230 protruding from an outer surface 211 of the base element 210 (e.g., at least proximate to the distal end 204).

The alternating materials of the medical apparatus 200 may be manufactured in any suitable way. For example, the medical apparatus 100 may be constructed using additive manufacturing as described in U.S. Pat. App. No. 62/927,092, entitled “ADDITIVE MANUFACTURING FOR MEDICAL DEVICES,” which is herein incorporated by reference. Specifically, the alternating materials may be formed by pixel-by-pixel 3D printing, using multiple print heads.

Further, as shown in FIG. 3, the medical apparatus 200 may include multiple portions of splines 230 defining and having different materials. For example, the at least one spline 230 may include a first longitudinal section 232 extending along the longitudinal direction 201 (e.g., including a first material) and a second longitudinal section 234 extending along the longitudinal direction 201 (e.g., including a second material). The first longitudinal section 232 may be proximate or adjacent to the second longitudinal section 234 (e.g., such that the second longitudinal section 234 is immediately before or after the first longitudinal section 232 along the longitudinal direction 201). In one or more embodiments, the first material (of the first longitudinal section 232) may be different than the second material (of the second longitudinal section 234). Therefore, the characteristics of the first longitudinal section 232 may be different than the second longitudinal section 234. For example, the first material may define a first Shore durometer that is different than a second Shore durometer of the second material. The differing hardness of adjacent portions of the at least one spline 230 may provide the ability to customize characteristics of the apparatus 200 for a particular application.

For example, the apparatus 200 may be customized for a particular flexure (e.g., bending) and torque transfer. Specifically, in certain applications, alternating the pattern of hard and soft polymers may, for example, assist in tracking the anatomy better, provide for increased torque transfer (as compared to soft polymer alone at the distal end), and not stretch excessively when pulling the catheter through tortuous anatomy. Further, an alternating pattern may be a gradient or may be a more discrete transition. For example, a hard/soft continuous spiral spline may have enough flexibility due to the soft segments to manage acute changes in vessel shape and direction while maintaining continuous engagement with the vessel wall through the hard segments of the spline to transfer torque to the irregular-shaped vessel wall. Further, the alternating hard/soft spiral splines may transfer more force to the vessel wall through very localized engagement (e.g., the integration of those small but optimized engagements pull the catheter through the tortuous section when rotating-to-advance).

Also, as shown in FIG. 4, the medical apparatus 200 may include multiple portions of the base element 210 defining and having different materials. For example, the base element 210 may include a first portion 212 extending along the longitudinal direction 201 and may include (e.g., be formed of) a first material. The base element 21 may also include a second portion 214 extending along the longitudinal direction 201 and may include (e.g., be formed of) a second material. In one or more embodiments, the first material (of the first portion 212) may be different than the second material (of the second portion 214). Therefore, the characteristics of the first portion 212 may be different than the second portion 214. For example, the first material may define a first Shore durometer that is different than a second Shore durometer of the second material. The differing hardness of adjacent portions of the base element 210 may assist in flexure and torque transfer, as described herein. In one or more embodiments, a continuous spiral spline may be more beneficial in continuous smooth vessels. In one or more embodiments, the at least one spline 230 may include (e.g., be formed of) a third material that is different than the first and second materials.

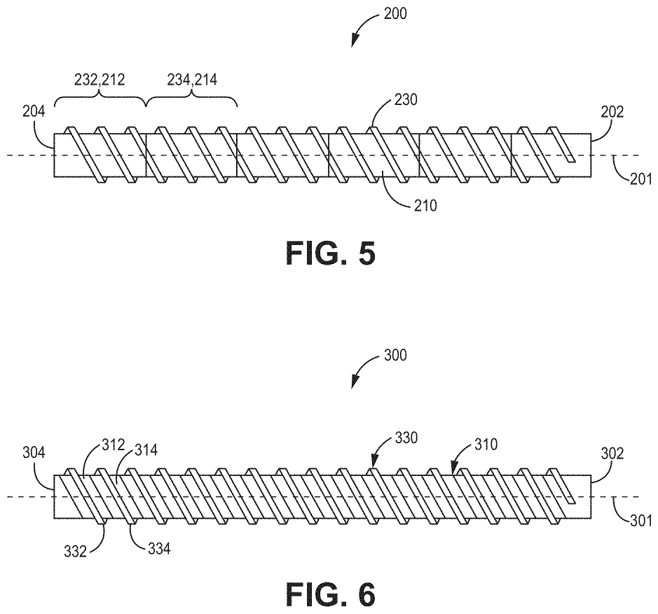

In one or more embodiments, for example as shown in FIG. 5, both the base element 210 and the at least one spline 230 may vary or alternate materials. For example, the first longitudinal section 232 may correspond or line up with the first portion 212 and the second longitudinal section 234 may correspond or line up with the second portion 214. Both of the first longitudinal section 232 and the first portion 212 may include (e.g., be formed of) a first material and both of the second longitudinal section 234 and the second portion 214 may include (e.g., be formed of) a second material. Using different materials to may assist in flexure and torque transfer, as described herein.

FIG. 6 illustrates yet another embodiment of alternating or varying materials of a medical apparatus 300. Specifically, the base element 310 may include a first helical base portion 312 extending along a helical path and a second helical base portion 314 extending along a helical path that is adjacent or next to the first helical base portion 312. The first helical base portion 312 may include (e.g., be formed of) a first material and the second helical base portion 314 may include (e.g., be formed of) a second material. Further, the at least one spline 330 may include a first spline 332 including (e.g., formed of) the first material and protruding from the first helical base portion 312. Also, in one or more embodiments, the at least one spline 330 may include a second spline 334 including (e.g., formed of) the second material and protruding from the second helical base portion 314. The alternating materials (e.g., of differing hardness) may provide the medical apparatus 100 with improved flexure and torque transfer (e.g., more continuous flexural properties along a given helical portion). Additionally, loading one spline with steroid or hydrophilic materials but not the other spline may help define the characteristics of each. Further, in one or more embodiments, one spline may be conductive (e.g., loaded with carbon black) and more compliant to the tissue, while the other spline is more structural and serves to pull the tip of the apparatus via rotate-to-advance such that the electrically active spiral spline may then be anchored to the vessel.

By altering one or both of the materials forming the base elements 210, 310 and the materials forming the at least one spline 230, 330, the apparatus 200, 300 may be customized for a specific application. For example, the materials forming the apparatus 200, 300 may define a specific torque to flexure ratio for the specific application (e.g., rigidity to apply a sufficient torque, yet flexible to navigate a tortious path for that application).

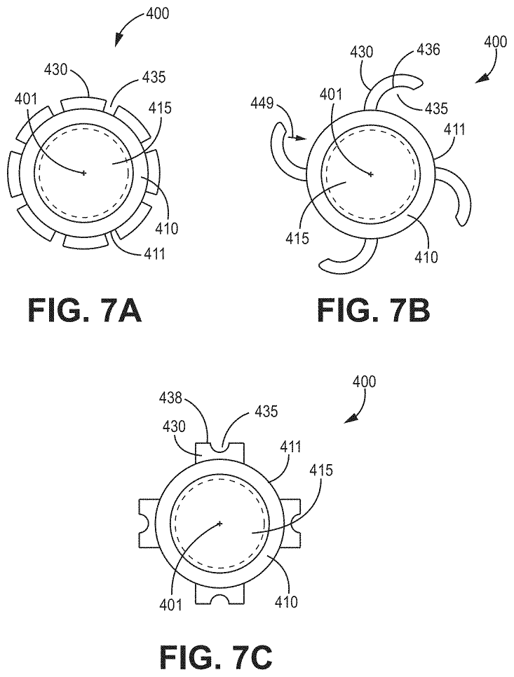

As shown in FIGS. 7A-7C, medical apparatus 400 may include various recesses or channels that may assist in allowing fluids to flow past the medical apparatus 400. The medical apparatus 400 may include the same components and characteristics as described pertaining to medical apparatus 100 illustrated in FIGS. 1-2. For example, the medical apparatus 400 may include a base element 410 extending along a longitudinal direction 401 between a distal end and a proximal end, and the base element 410 may define a lumen 415 therein. Also, similar to medical apparatus 100 illustrated in FIGS. 1-2, the medical apparatus 400 may include at least one spline 430 protruding from an outer surface 411 of the base element 410 (e.g., at least proximate to the distal end).

Additionally, FIG. 7A illustrates the at least one spline 430 defining a fluid flow channel 435 extending between splines 430. In other words, the fluid flow channel 435 may be defined between two separated splines 430 and the outer surface 411 of the base element 410. The fluid flow channel 435 may extend along the longitudinal direction 401. The at least one spline 430 may be configured to protect the fluid flow channel 435 from tissue intrusion (e.g., from the body of a patient) into the fluid flow channel 435 when the apparatus 400 is implanted. Specifically, due to the tortious path in which the medical apparatus 400 may be implanted in the body, it may be beneficial to have dedicated fluid flow paths that allow the passage of fluids (e.g., to maintain proper hydration) and may be designed to restrict kinking or obstruction.

Further, the fluid flow channel 435 may be constructed based on the specific therapy performed by the medical apparatus 400 or the anatomy to be traversed by the medical apparatus 400. For example, a specific therapy may include tunneling through the ventricular septum and another specific therapy may advance a delivery catheter deep into the coronary venous system through tortuous branches that get smaller and smaller in dimension as the delivery catheter is advanced deeper.

In one or more embodiments, the at least one spline 430 may be configured to deflect toward the outer surface 411 of the base element 410 when the apparatus 400 is implanted, e.g., as indicated by arrow 449 shown in FIG. 7B. The at least one spline 430 may deflect such that a surface 436 of the at least one spline 430 defines a fluid flow channel 435 between the at least one spline 430 and the outer surface 411 of the base element 410 (e.g., causing the at least one spline 430 to “tent”).

In one or more embodiments, the at least one spline 430 may define an outermost surface 438 (e.g., in a radial direction) and the fluid flow channel 435 may be defined by a recess in the outermost surface 438, e.g., as shown in FIG. 7C. In one or more embodiments, a hydrophilic material may be disposed in any suitable location on the at least one spline 430 to assist in the flow of fluid (e.g., within the fluid flow channel 435). For example, the hydrophilic material may include a discrete layer, e.g., within, proximate to, layered on, coated, etc., and partially filling the fluid flow channel 435. Specifically, the hydrophilic material may be disposed in the recess of the at least one spline 430, on the outermost surface 438 of the at least one spline 430, and/or the surface 436 of the at least one spline 430.

In one or more embodiments, fluid flow channel 435 may have an extended surface to increase attachment sites or the fluid flow channel 435 may incorporate a matrix to assist in holding the cross-linked hydrogel or hydrophilic material in the fluid flow channel 435 (e.g., to increase the durability of the hydrophilic coating). Further, ozone, corona, or plasma surface pre-treatment may be used to improve covalent bonding to the substrate. Some hydrophilic coatings may be cured using ultraviolet light after being deposited on the surface. The hydrophilic coating deposition may be preceded by printing a tie-layer material miscible to the substrate and to the hydrophilic material for improved durability or to attach the hydrophilic material to a hydrophobic substrate material. In one or more embodiments, two types of hydrophilic materials may be printed onto the surface (e.g., of the fluid flow channel 435), with one hydrophilic material printed on a first surface of the fluid flow channel 435 and the other one printed on a second surface of the fluid flow channel 435 that is different from the first surface. The hydrophilic material in the fluid flow channel 435 may swell more and provide fluid in high contact stress locations to flow towards a more durable hydrophilic material which does not swell as much.

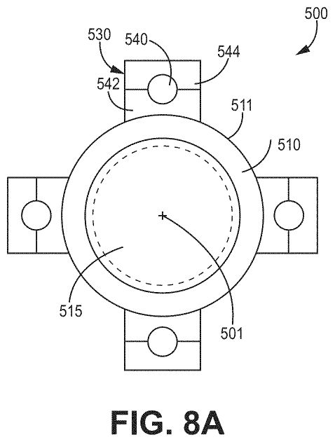

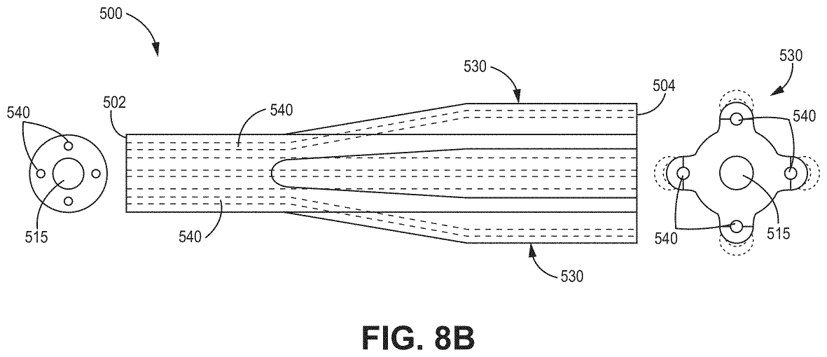

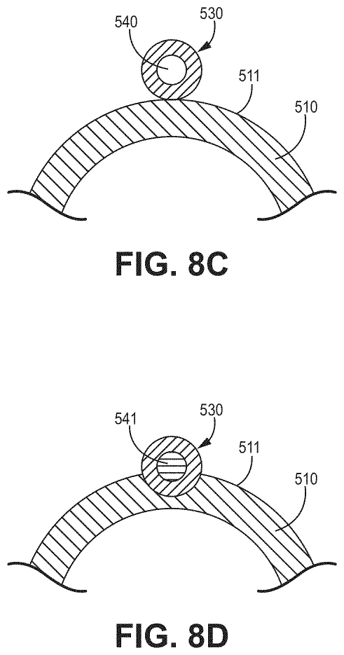

As shown in FIG. 8A, medical apparatus 500 may be configured to be inflated to assist with movement of the medical apparatus 500 or with a specific procedure. The medical apparatus 500 may include the same components and characteristics as described pertaining to medical apparatus 100 illustrated in FIGS. 1-2. For example, the medical apparatus 500 may include a base element 510 extending along a longitudinal direction 501 between a distal end and a proximal end, and the base element 510 may define a lumen 515 therein. Also, similar to medical apparatus 100 illustrated in FIGS. 1-2, the medical apparatus 500 may include at least one spline 530 protruding from an outer surface 511 of the base element 510 (e.g., at least proximate to the distal end).

The at least one spline 530 may define a spline lumen 540 extending through the at least one spline 530. Further, the at least one spline 530 may include a flexible material (e.g., flexible relative to the remainder of the apparatus 500) such that the at least one spline 530 may be inflatable (e.g., by inserting fluid through the spline lumen 540). In one or more embodiments, the at least one spline 530 may include a first portion 542 formed from a first material and a second portion 544 formed from a second material. The second portion 544 may be disposed radially distal to the first portion 542 (e.g., the second portion is outside of or exterior to the first portion). The first material may define a first Shore durometer and the second material may define a second Shore durometer less than the first Shore durometer. Therefore, the outermost material (e.g., the second portion 544) of the at least one spline 530 may allow for the at least one spline 530 to inflate when a fluid is inserted into the spline lumen 540. In other embodiments, the first and second materials may be formed from the same flexible material.

The at least one spline 530 may be manufactured to be inflatable in any suitable way. For example, the medical apparatus 500 may be constructed using additive manufacturing as described in U.S. Pat. App. No. 62/927,092, entitled “ADDITIVE MANUFACTURING FOR MEDICAL DEVICES,” which is herein incorporated by reference, and the at least one spline 530 may include further processing. For example, in one or more embodiments, a thin wall of highly elastic material, such as a biocompatible elastomer, may be extruded over a core rod (e.g., made of copper). The biocompatible elastomer may be placed through an extruder longitudinal for each spline of the at least one spline 530. The biocompatible elastomer may then be moved through either a die cut out for the at least one spline 530 or through a die with no cut outs (e.g., no splines). During a second pass, a second material may be placed, or formed, thereon (e.g., if the proximal and medial portions are not to be inflatable) and transitioned to a biocompatible elastomer for the portion of the at least one spline 530 that is inflatable (e.g., the balloon portion of the at least one spline 530). The core rod may then be stretched and pulled out of the middle of the at least one spline 530, forming a lumen. Next, a distal tip may be mold inserted to close off the lumen of the at least one spline 530. Also, a hub may be mold inserted to create an inflation manifold in fluid communication with the lumen (e.g., therefore using a Luer fitting to attach an inflator or a syringe).

In another embodiment, the at least one inflatable spline 530 may be manufactured using pixel-by-pixel 3D printing. In such embodiments, the at least one spline 530 may include a post-printing cure, sealer, or binder. Further, if the at least one inflatable spline 530 is created by multiple passes, tie layers may be used, having potentially different size pixels (e.g., different shot sizes), etc.

Further, in one or more embodiments, the base element 510 may include a flexible material such that the base element 510 is inflatable (e.g., the apparatus 500 may not include support element such as a braid or reinforcement underneath the base element 510 at the inflatable portion). In such embodiments, the at least one spline 530 may be formed from a flexible material such that the at least one spline is expandable (e.g., when the base element 510 is inflated).

The inflatable spline 530 (e.g., as shown in FIG. 8A) may assist in providing perfusion while dilating and/or anchoring the vessel or anatomy because the inflatable spline 530 may not occlude the vessel (e.g., as compared to a conventional inflatable catheter balloon). Further, the inflatable spline 530 may allow for atraumatic rotate-to-advance by advancing with the spiral splines 530 deflated for one turn, inflating and dilating while stationary, and repeating. Also, there may be variants of this incremental advancing of a catheter or lead, and may apply to advancing a catheter or lead in a use condition where it is not in a vessel, e.g., the ventricular septum.

In one or more embodiments, the spline lumen 540 (e.g., shown in broken lines) may extend from a proximal end 502 to the distal end 504, e.g., as shown in FIG. 8B. Each spline lumen 540 may extend up to a circular manifold, which is common to the proximal end 102 of the inflatable spiraling spline lumen 540. Further, the spline lumen 540 may transition from the base element 510 and into each of the splines 530.

Further, in one or more embodiments, the apparatus 500 may define a tubular filament spline 530 that may be wound onto the base element 510, e.g., as shown in FIGS. 8C and 8D. For example, the tubular filament spline 530 may be compliant to the surrounding body tissue (e.g., when in use) and may be more atraumatic than a spline that is solid throughout or “filled.” The spline 530 may be positioned on and coupled to an outer surface 511 of the base element 510 (e.g., as shown in FIG. 8C) or may be partially or completely embedded within the base element 510 (e.g., as shown in FIG. 8D). Additionally, if the spline 530 is made of an elastic material (e.g., as described herein), the spline 530 may be inflatable via the lumen 540 extending therethrough (e.g., the portion of the spline 530 that is not embedded may inflate and expand from the base element 510). In one or more embodiments, a removable core rod/wire 541 may be inserted into the lumen 540 when forming or manufacturing the lumen 540 (and, e.g., removed therefrom after manufacturing and prior to use), as described herein. In such embodiments, the core rod/wire 541 may include filaments or monofilaments such as, for example, Ultra-High Molecular Weight Polyethylene (UHMWPE, e.g., Dyneema® manufactured by Dutch State Mines), Polytetrafluoroethylene (PTFE), Fluorinated Ethylene Propylene (FEP), etc. Specifically, the core rode/wire 541 may define a low coefficient of friction fiber (e.g., UHMWPE), may not have the rigidity of a typical core rod used in polymer processing (e.g., flexible to more easily navigate spiraled splines during removal), and/or may include a stranded wire cable.

It is noted that the embodiments illustrated in FIG. 8C may be manufactured through a process that is a variant of Extruded Parallel Tubing (i.e., Paratubing). Paratubing includes arranging two conventional extruders and outputting material in parallel (and, e.g., defining lumens through each). However, the typical Paratubing manufacturing process may not be suitable for wrapping or spiraling the smaller extrusion (e.g., defining the tubular spline) around a large lumen (e.g., defining the base element). Therefore, the process described herein provides a process for manufacturing the apparatus 500, which may define a tubular filament spline 530 spiraled or wound onto the base element 510 as shown in FIG. 8C.

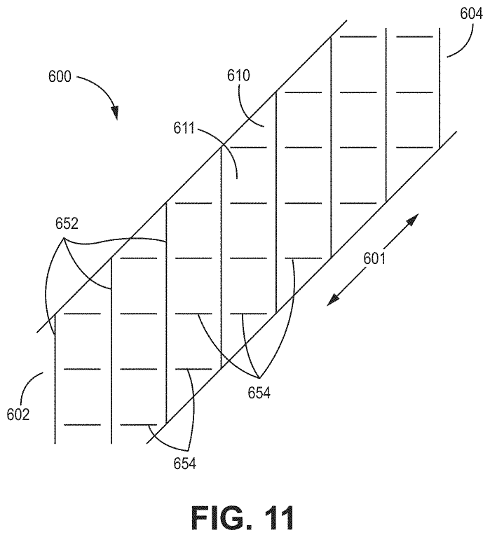

As shown in FIGS. 9-11, the medical apparatus 600 may include multiple splines that extend along paths that cross or intersect on an outer surface 611. The medical apparatus 600 may include the same components and characteristics as described pertaining to medical apparatus 100 illustrated in FIGS. 1-2. For example, the medical apparatus 600 may include a base element 610 extending along a longitudinal direction 601 between a distal end 604 and a proximal end 602, and the base element 610 may define a lumen therein. Also, similar to medical apparatus 100 illustrated in FIGS. 1-2, the medical apparatus 600 may include at least one spline 630 protruding from the outer surface 611 of the base element 610 (e.g., at least proximate to the distal end 604).

The at least one spline 630 may include a first spline 652 extending along a first helical path and a second spline 654 extending along a second helical path. The second helical path may be defined as extending in an opposite rotational direction than the first helical path such that the path of the first spline 652 and the path of the second spline 654 intersect. In one embodiment, the first and second splines 652, 654 may contact and intersect.

However, in another embodiment, the first and second splines 652, 654 do not physically contact, but the paths upon which the first and second splines 652, 654 extend cross one another. Therefore, in this embodiment, the first spline 652 may be described as continuous, while the second spline 654 may be described as not continuous or segmented.

In one or more embodiments, the first spline 652 may protrude from the outer surface 611 in a radial direction by a first distance 653 that is greater than a second distance 655 that the second spline 654 protrudes from the outer surface 611 (e.g., as shown in FIG. 10).

In one or more embodiments, the first spline 652 may be configured to assist in controlling movement of the apparatus 600 and the second spline 654 may be flexible in one direction and restrict specific movement, as described further herein. For example, the second spline 654 may protrude from the outer surface 611 at an angle towards the proximal end 602. Therefore, when the medical apparatus 600 (e.g., the distal end 604) is inserted into a patient, the second spline 654 may deflect towards the outer surface 611. However, when the medical apparatus 600 is moved in the opposite direction (e.g., towards the proximal end 602), the second spline 654 may engage the tissue within which the apparatus 600 is positioned to, e.g., restrict movement, etc.

Each of the first and second splines 652, 654 may include the same or different material. For example, the first spline 652 may include (e.g., be formed of) a first material and the second spline 654 may include (e.g., be formed of) a second material. In one or more embodiments, the first material may define a first Shore durometer that is less than a second Shore durometer of the second material.

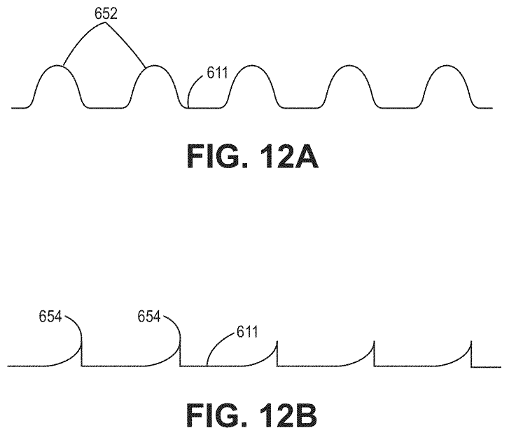

The medical apparatus including cross-spiraling splines (e.g., as shown in FIGS. 9-11) may be constructed in a variety of different ways. For example, the cross-spiraling splines may be produced using multiple additive steps, three-dimensional printing the cross-spiraling splines on the base element, taking material from a first spline to produce a second cross-spiraling spline, etc. For example, as shown in FIG. 12A, the first spline 652 may protrude from the outer surface 611 and define a dome-shape. Further, in a process in which a tool takes material from the first spline 652 to create or form the second spline 654, the second spline 654 may define an angled shape, as shown in FIG. 12B.

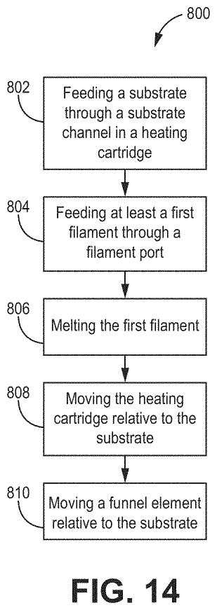

Specifically, FIG. 14 shows one example of a method 800 for additive manufacturing of an implantable medical catheter having cross-spiraling splines. For example, the method 800 may include feeding 802 a substrate through a substrate channel in a heating cartridge. The substrate channel may be in fluid communication with an interior cavity of the heating cartridge. Further, the method may include feeding 804 at least a first filament through a filament port into the interior cavity and melting 806 the first filament in the interior cavity. The method may then include moving 808 the heating cartridge relative to the substrate at least in a longitudinal direction and a first rotational direction to form a catheter jacket and a first spline protruding from an outer surface of the catheter jacket comprising material from the first filament. In other words, melting 806 the first filament may provide the material to create the catheter jacket and the spline and moving 808 the heating cartridge relative to the substrate may form the catheter jacket and the spline. By moving the heating cartridge in a rotational direction, the first spline may extend along a first helical path consistent with the rotational direction.

The method may also include moving 810 a funnel element relative to the substrate at least in a longitudinal direction and a second rotational direction opposite to the first rotational direction to form a second spline protruding from the outer surface of the catheter jacket comprising material from the first spline. In other words, the funnel element may move relative to the catheter jacket to “smear” the first material from the first spline to form the second spline. The second spline may extend along a second helical path extending in an opposite rotational direction than the first helical path such that the first spline and the second spline intersect.

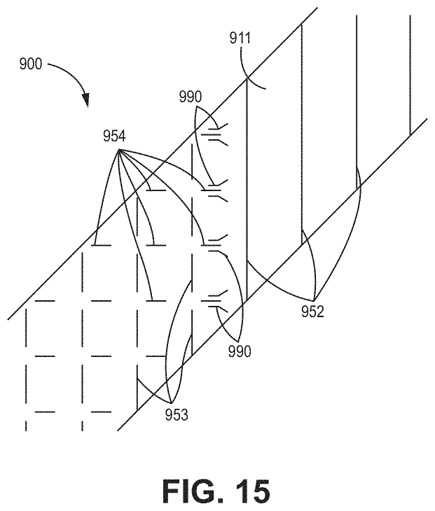

For example, FIG. 15 illustrates one embodiment of cross-spiraling splines extending along an outer surface 911 of a medical apparatus 900 project along a plane. Specifically, FIG. 15 illustrates an initial first spline 952 that may be formed along the outer surface 911 using a profiled extrusion die, which extends continuously along the outer surface 911. The second spline 954 may be formed by displacing a portion of the melted filament material of the initial first spline 952. Specifically, a funnel die 990 may move transverse to the initial first spline 952 such that the funnel die 990 collects melted filament material from the initial first spline that deposits a length of the filament material to form the crossing second spline 954. Thereafter, only a portion of the initial first spline 952 may remain such that the resulting first spline 953 is noncontiguous (e.g., because of a portion being used to form the second spline 954). After each of the first and second splines 953, 954 are formed, the filament material may harden or cure. It is noted that multiple funnel dies 990 are illustrated in FIG. 15, but any suitable number of funnel die 990 may be used.

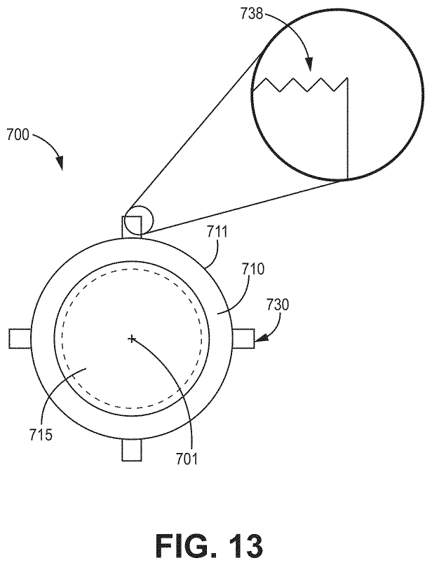

As shown in FIG. 13, medical apparatus 700 may include at least one spline 730 having or defining a textured surface. The medical apparatus 700 may include the same components and characteristics as described pertaining to medical apparatus 100 illustrated in FIGS. 1-2. For example, the medical apparatus 700 may include a base element 710 extending along a longitudinal direction 701 between a distal end and a proximal end, and the base element 710 may define a lumen 715 therein. Also, similar to medical apparatus 100 illustrated in FIGS. 1-2, the medical apparatus 700 may include at least one spline 730 protruding from an outer surface 711 of the base element 710 (e.g., at least proximate to the distal end).

Further, the at least one spline 730 may define a textured outermost surface 738 to facilitate implantation of the medical apparatus 700. For example, in one or more embodiments, the textured outermost surface 738 may define a plurality of dimples. Also, in one or more embodiments, the textured outermost surface 738 may define a cross-hatch. Further, in one or more embodiments, the textured outermost surface 738 may define a plurality of micro-grooves or micro-splines. It is noted that, while FIG. 13 illustrates only the outermost surface 738 as textured, any surface (such as a side surface) of the at least one spline 730 may define a textured surface.

### Illustrative Embodiments

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

A1. An apparatus comprising:
a base element extending along a longitudinal direction between a distal end and a proximal end, wherein the base element comprises a first material, wherein the base element defines a lumen therein; and
at least one spline protruding from an outer surface of the base element at least proximate to the distal end only partially covering the outer surface, wherein the at least one spline comprises a second material, wherein the first material defines a first Shore durometer that is different than a second Shore durometer of the second material.

A2. The apparatus of embodiment A1, wherein the at least one spline extends along a helical path on the outer surface of the base element.

A3. The apparatus of embodiment A1 or A2, wherein the at least one spline comprises a plurality of splines extending along the longitudinal direction.

A4. The apparatus of any preceding A embodiment, wherein the base element comprises at least one of a braided wire, inner liner.

B1. An apparatus comprising:
a base element extending along the longitudinal direction between a distal end and a proximal end, wherein the base element comprises a first material, wherein the base element defines a lumen therein; and
at least one spline protruding from an outer surface of the base element at least proximate to the distal end, wherein the at least one spline comprises a second material, wherein the second material comprises a bioresorbable material.

B2. The apparatus of embodiment B1, wherein the second material comprises a steroid material.

B3. The apparatus embodiment B1 or B2, wherein the second material comprises a drug and a polymer.

B4. The apparatus of any preceding B embodiment, wherein the second material comprises a drug in a porous silicone material.

C1. An apparatus comprising:
a base element extending along the longitudinal direction between a distal end and a proximal end, wherein the base element comprises a first material, wherein the base element defines a lumen therein; and
at least one spline protruding from an outer surface of the base element at least proximate to the distal end, wherein the at least one spline comprises a second material, wherein the second material comprises a steroid material.

D1. An apparatus comprising:
a base element extending along a longitudinal direction between a distal end and a proximal end, wherein the base element defines a lumen therein; and
at least one spline protruding from an outer surface of the base element at least proximate to the distal end, wherein the at least one spline comprises a first longitudinal section extending along the longitudinal direction comprising a first material and a second longitudinal section extending along the longitudinal direction comprising a second material, wherein the first longitudinal section is proximate or adjacent to the second longitudinal section.

D2. The apparatus of embodiment D1, wherein the at least one spline extends along a helical path on the outer surface of the base element.

D3. The apparatus of embodiment D1 or D2, wherein the at least one spline comprises a plurality of splines extending along the longitudinal direction.

D4. The apparatus of any preceding D embodiment, wherein the first material defines a first Shore durometer that is different than a second Shore durometer of the second material.

E1. An apparatus comprising:
a base element extending along a longitudinal direction between a distal end and a proximal end, wherein the base element comprises a first portion extending along the longitudinal direction comprising a first material and a second portion extending along the longitudinal direction comprising a second material, wherein the first portion extends proximate or adjacent to the second portion, wherein the base element defines a lumen therein; and
at least one spline protruding from an outer surface of at least one of the first portion and the second portion at least proximate to the distal end.

E2. The apparatus of embodiment E1, wherein the at least one spline extends along a helical path on the outer surface of the base element.

E3. The apparatus of embodiment E1 or E2, wherein the at least one spline comprises a plurality of splines extending along the longitudinal direction.

E4. The apparatus of any preceding E embodiment, wherein the first material defines a first Shore durometer that is different than a second Shore durometer of the second material.

E5. The apparatus of any preceding E embodiment, wherein the at least one spline comprises: a first spline comprising the first material and protruding from the outer surface of the first portion; and optionally a second spline comprising the second material and protruding from the outer surface of the second portion.

E6. The apparatus of any preceding E embodiment, wherein the first portion and the second portions extend along a helical path.

E7. The apparatus of any preceding E embodiment, wherein the at least one spline comprises a third material that is different than the first and second materials.

F1. An apparatus comprising:
a base element extending along a longitudinal direction between a distal end and a proximal end, wherein the base element defines a lumen therein; and
at least one spline protruding from an outer surface of the base element at least proximate to the distal end, wherein the at least one spline defines a fluid flow channel extending along the longitudinal direction, wherein the at least one spline is configured to protect from tissue intrusion into the fluid flow channel when the apparatus is implanted.

F2. The apparatus of embodiment F1, wherein the at least one spline is configured to deflect toward the outer surface of the base element when the apparatus is implanted such that a surface of the at least one spline defines the fluid flow channel between the at least one spline and the outer surface of the base element.

F3. The apparatus of embodiment F1 or F2, wherein the at least one spline defines an outermost surface in a radial direction and the fluid flow channel is defined by a recess in the outermost surface.

F4. The apparatus of embodiment F3, further comprising a hydrophilic material disposed in the recess of the at least one spline.

F5. The apparatus of any preceding F embodiment, further comprising a hydrophilic material disposed on the at least one spline.

G1. An apparatus comprising:
a base element extending along a longitudinal direction between a distal end and a proximal end, wherein the base element defines a lumen therein; and
at least one spline protruding from an outer surface of the base element at least proximate to the distal end, wherein the at least one spline defines a spline lumen extending through the at least one spline, wherein the at least one spline comprises a flexible material such that the at least one spline is inflatable.

G2. The apparatus of embodiment G1, wherein the at least one spline comprises a first portion comprising a first material defining a first Shore durometer and a second portion comprising the flexible material defining a second Shore durometer less than the first Shore durometer, wherein the second portion is disposed radially distal to the first portion.

G3. The apparatus of embodiment G1 or G2, wherein the base element comprises a first material defining a first Shore durometer and the flexible material defining a second Shore durometer less than the first Shore durometer.

H1. An apparatus comprising:
a base element extending along a longitudinal direction between a distal end and a proximal end, wherein the base element defines a lumen therein, wherein the base element comprises a flexible material such that the base element is inflatable; and
at least one spline protruding from an outer surface of the base element at least proximate to the distal end, wherein the at least one spline comprises a flexible material such that the at least one spline is expandable.

I1. An apparatus comprising:
a base element extending along a longitudinal direction between a distal end and a proximal end, wherein the base element defines a lumen therein; and
at least one spline protruding from an outer surface of the base element at least proximate to the distal end, wherein the at least one spline comprises a first spline extending along a first helical path and a second spline extending along a second helical path extending in an opposite rotational direction than the first helical path such that the first spline and the second spline intersect.

I2. The apparatus of embodiment I1, wherein the first spline protrudes from the outer surface in a radial direction by a first distance greater than a second distance the second spline protrudes from the outer surface.

I3. The apparatus of embodiment I1 or I2, wherein the second spline protrudes from the outer surface in a direction towards the proximal end.

I4. The apparatus of any preceding I embodiment, wherein the first spline comprises a first material and the second spline comprises a second material, the first material defining a first Shore durometer that is less than a second Shore durometer of the second material.

J1. A method comprising:
feeding a substrate through a substrate channel in a heating cartridge, the substrate channel in fluid communication with an interior cavity of the heating cartridge;
feeding at least a first filament through a filament port into the interior cavity;
melting the first filament in the interior cavity;
moving the heating cartridge relative to the substrate at least in a longitudinal direction and a first rotational direction to form a catheter jacket and a first spline protruding from an outer surface of the catheter jacket comprising material from the first filament, wherein the first spline extends along a first helical path; and
moving a funnel element relative to the substrate at least in a longitudinal direction and a second rotational direction opposite to the first rotational direction to form a second spline protruding from the outer surface of the catheter jacket comprising material from the first spline, wherein the second spline extends along a second helical path extending in an opposite rotational direction than the first helical path such that the first spline and the second spline intersect.

K1. An apparatus comprising:
a base element extending along a longitudinal direction between a distal end and a proximal end, wherein the base element defines a lumen therein; and
at least one spline protruding from an outer surface of the base element at least proximate to the distal end, wherein the at least one spline defines a textured outermost surface to facilitate implantation.

K2. The apparatus of embodiment K1, wherein the textured outermost surface defines a plurality of dimples.

K3. The apparatus of embodiment K1 or K2, wherein the textured outermost surface defines a cross-hatch.

K4. The apparatus of any preceding K embodiment, wherein the textured outermost surface defines a plurality of microgrooves.

Thus, various embodiments of medical devices including at least one spline extending from an outer surface of the device and along a longitudinal direction and methods to manufacture the same are disclosed. It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are expressly incorporated herein by reference in their entirety for all purposes, except to the extent any aspect directly contradicts this disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

The singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

1. An apparatus comprising:
a base element extending along a longitudinal direction between a distal end and a proximal end, wherein the base element comprises a first material, wherein the base element defines a lumen therein; and
at least one spline protruding from an outer surface of the base element at least proximate to the distal end only partially covering the outer surface, wherein the at least one spline comprises a second material, wherein the first material defines a first Shore durometer that is different than a second Shore durometer of the second material.

2. The apparatus of claim 1, wherein the at least one spline extends along a helical path on the outer surface of the base element.

3. The apparatus of claim 1, wherein the at least one spline comprises a plurality of splines extending along the longitudinal direction.

4. The apparatus of claim 1, wherein the base element comprises at least one of a braided wire and an inner liner.

5. The apparatus of claim 1, wherein the second material comprises a bioresorbable material.

6. The apparatus of claim 1, wherein the second material comprises a steroid material.

7. The apparatus of claim 1, wherein the second material comprises a drug and a polymer.

8. The apparatus of claim 1, wherein the second material comprises a drug in a porous silicone material.

9. An apparatus comprising:
a base element extending along a longitudinal direction between a distal end and a proximal end, wherein the base element defines a lumen therein; and
at least one spline protruding from an outer surface of the base element at least proximate to the distal end, wherein the at least one spline comprises a first longitudinal section extending along the longitudinal direction comprising a first material and a second longitudinal section extending along the longitudinal direction comprising a second material, wherein the first longitudinal section is proximate or adjacent to the second longitudinal section.

10. The apparatus of claim 9, wherein the at least one spline extends along a helical path on the outer surface of the base element.

11. The apparatus of claim 9, wherein the at least one spline comprises a plurality of splines extending along the longitudinal direction.

12. The apparatus of claim 9, wherein the first material defines a first Shore durometer that is different than a second Shore durometer of the second material.

13. The apparatus of claim 9, wherein at least one of the first material and the second material comprises a bioresorbable material.

14. The apparatus of claim 9, wherein at least one of the first material and the second material comprises a steroid material.

15. An apparatus comprising:
a base element extending along a longitudinal direction between a distal end and a proximal end, wherein the base element comprises a first portion extending along the longitudinal direction comprising a first material and a second portion extending along the longitudinal direction comprising a second material, wherein the first portion extends proximate or adjacent to the second portion, wherein the base element defines a lumen therein; and
at least one spline protruding from an outer surface of at least one of the first portion and the second portion at least proximate to the distal end.

16. The apparatus of claim 15, wherein the at least one spline extends along a helical path on the outer surface of the base element.

17. The apparatus of claim 15, wherein the at least one spline comprises a plurality of splines extending along the longitudinal direction.

18. The apparatus of claim 15, wherein the first material defines a first Shore durometer that is different than a second Shore durometer of the second material.

19. The apparatus of claim 15, wherein the at least one spline comprises:
a first spline comprising the first material and protruding from the outer surface of the first portion; and
a second spline comprising the second material and protruding from the outer surface of the second portion.

20. The apparatus of claim 15, wherein the first portion and the second portions extend along a helical path.
